SEQUENCE LISTING (3 sequences)

SEQ ID NO: 1 - protein, 9 aa, Human Herpesvirus 4 (HHV-4)
NGGVMKEKD

SEQ ID NO: 2 - protein, 7 aa, Human Herpesvirus 4 (HHV-4)
GKNDKEE

SEQ ID NO: 3 - protein, 8 aa, Human Herpesvirus 4 (HHV-4)
WFKLGPGE

(19) United States
(12) Patent Application Publication
(10) Pub. No.: US 2011/0135642 A1
(43) Pub. Date: Jun. 9, 2011

(54) Pharmaceutical Compositions Comprising Antibodies Binding To EBV (Ebstein-Barr Virus) Protein BARF1

(75) Inventor: Tadamasa Ooka, Lyon (FR)

(73) Assignees: UNIVERSITE CLAUDE BERNAND LYON 1, Villeurbanne (FR); Centre National De La Recherche Scientifiue (CNRS), Paris (FR)

(21) Appl. No.: 13/057,721

(22) PCT Filed: Aug. 7, 2009
(86) PCT No.: PCT/EP09/60275

§ 371 Date: Feb. 4, 2011

(30) Foreign Application Priority Data
Aug. 8, 2008 (FR) 08162086.6

(51) Int. Cl. A61K 39/42; A61K 35/00; A61K 31/7088; C07K 16/08; A61K 38/08; A61P 35/00; A61P 31/22

(52) U.S. Cl. 424/133.1; 424/139.1; 424/93.2; 514/44 R; 530/387.9; 514/4.2

(57) ABSTRACT

The invention relates to pharmaceutical and vaccine compositions comprising an antibody binding specifically to selected peptides of EBV protein BARF1.

The present invention relates to compositions for use in immunotherapy and vaccination comprising antibodies binding to BARF1 or peptides derived from BARF1.

The Epstein-Barr virus (EBV) is associated with several human cancers: Nasopharyngeal carcinoma, Gastric carcinoma, Burkitt's lymphoma, Hodgkin's lymphoma, lymphoma induced in AIDS patients, Esophage and Intrahepatic cholangiocarcinoma. Recent data showed that EBV is also implicated in nasal NK/T-cell lymphoma and intra-hepatic cholangiocarcinoma. Oral hairy leucoplasia (OHL), frequent in AIDS patients is also tigtly associated with EBV. EBV is therefore both lymphotropic and epitheliotropic.

Several therapeutic methods for EBV-related cancers have been used including radio- and chemo-therapy. However radio- and chemotherapy pose classical problems (toxicity, dose, etc.). Several cellular and viral gene therapies have also been developed which are generally based on viral and/or cellular proteins as targets. However, these therapies have not performed sufficiently well.

In immunotherapy, anti-EGFR antibodies (Epidermal Growth Factor Receptor) were also proposed, particularly for treatment of carcinomas (NPC, Thymomes, Lung, Cervical carcinoma, Colon, Breast, and Head and Neck), because epithelial tumor cells associated or not with EBV express EGFR. The treatment is therefore not exclusive for EBV-associated carcinomas. Efficiency of the treatment (monoclonal antibody Cetumximab) is being evaluated for cervical cancer and thymoma. However, there is a risk that patients treated with anti-EGFR in combination with radiotherapy become radio-resistant.

Nasopharyngeal carcinoma (NPC) is a human malignancy derived from the epithelium of the retro-nasal cavity. It is one of the most striking examples of a human malignancy that is consistantly associated with a virus. The full-length genome of Epstein-Barr Virus (EBV) is contained in all malignant NPC cells and it encodes viral proteins that probably contribute to the malignant phenotype (Decaussin G, Sbih-Lammali F, De Turenne-Tessier M, Bougermouh A M, Ooka T. 2000. Cancer Res 60: 5584-5588; Ooka T: 2005. In. Epstein-Barr Virus. Horizon Press, Annette Griffin: Edited by Erle S. Robertson. Chapter 28: p.p 613-630). Even though EBV infection is ubiquitous in humans, the incidence of NPC is extremely variable depending on the geographic area.

NPC biopsies expressed consistently several EBV genes in including genes encoding EBERs, EBNA1, LMP1, LMP2A, BARF0 and BARF1. Among them, only LMP1 and BARF1 are capable of inducing malignant transformation in rodent fibroblasts (Wei and Ooka, 1989, EMBO J. 8 :2897-903; Wang D, Liebowitz D and Kieff E. 1985. Cell 43:831-840). A large portion (>98%) of NPC biopsies expressed BARF1 (Decaussin G, Sbih-Lammali F, De Turenne-Tessier M, Bougermouth A M, Ooka T. 2000 Cancer Res. 60: 5584-8; Hayes D P, Brink A A, Vervoort M B, Middeldorp J M, Meijer C J, van den Brule A. 1999 J. Mol. Pathol. 52: 97-103.). Moreover, 5-10% of gastric carcinoma in the world are associated with EBV infection and almost all EBV-associated carcinoma's biopsy express BARF1. Among the viral lytic proteins, BARF1 alone was expressed consistently and at high levels in NPC- and EBV-associated GC carcinoma as well as in EBV-immortalized epithelial cells in vitro (Ooka, 2005, Chapter 28, In. Epstein-Barr Virus. Horizon Press, Annette Griffin: Edited by Erle S. Robertson. Page 613-630). On the other hand, BARF1 was able to immortalize in vitro primary monkey kidney epithelial cells (Wei M X, de Turenne-Tessier M, Decaussin G, Benet G and Ooka T. 1997 Oncogene 14: 3073-3082).

BARF1 as an oncogene encoded by EBV was discovered by Wei and Ooka in 1989 (Wei and Ooka, 1989, EMBO J. 8: 2897-903). BARF1 oncogene has both transforming and immortalising activity when expressed in rodent fibroblast (Wei and Ooka, 1989, EMBO J. 8: 2897-903), human B cell (Wei M X, Moulin, J C, Decaussin G, Berger F, Ooka T. 1994, Cancer Res. 54: 1843-8), human epithelial cell line and primary primate epithelial cells (Wei M X, de Turenne-Tessier M, Decaussin G, Benet G, Ooka T. 1997, Oncogene, 14: 3073-3082). BARF1 is also expressed in lymphoma induced in Tamarin (New world monkey) after injection of EBV particles (Zhang C X, Decaussin G, Finerty S, Morgan A, Ooka T. 1992, Virus Res 26: 153-166.) BARF1 activates Bcl2, c-myc, CD23, CD21, transferrin receptor and several transcriptional factors (Ooka, 2005, Chapter 28, In. Epstein-Barr Virus. Horizon Press, Annette Griffin: Edited by Erle S. Robertson. Page 613-630). BARF1 activates cyclin D1 in transfected cells and in EBV-associated gastric carcinoma cells (Wiech, T., Nikolopoulos, E., Lassmann, S., Heidt, T., Sarbia, M., Werner, M., Shimizu, Y., Sakka, M., Ooka, T and Zur Hausen. A. 2008, Virchows Archiv 452 :621-628). BARF1 activates the cell cycle when transfected in epithelial cells and this sequence was found in the <<Double Minute>> chromosomes, that are frequently observed in cancer cells containing amplified cellular genes like c-myc and mdm2 (Karran L., Teo C. G., King D., Hitt M. M., Gao Y., Wedderburn N. and Griffin B. E., 1990. Int. J. Cancer 45, 763-772). BARF1 protein is secreted by latently infected B cells (Fiorini and Ooka, 2008, Virology Journal, 5:70). This suggests that BARF1 gene belongs to the latent family of genes. A transforming domain was localised in the N-terminal region of BARF1 protein including 1-56th a.a which was also implied in the activation of anti-apoptotic Bcl2 (Sheng W, Decaussin G, Sumner S, Ooka T. 2001, Oncogene 20: 1176-1185).

BARF1 likely plays an important role in epithelial oncogenesis. BARF1 protein showed hexamer oligomeric structure determined by crystallography study (Tarbouriech N, Ruggiero F, de Turenne-Tessier M, Ooka T, Burmeister W P. 2006, J Mol Biol. 359 :667-678.) and acts as a powerful mitogene (Sall A, Caserta S, Jolicoeur P, Franqueville L, de Turenne-Tessier M and Ooka T. 2004. Oncogene. 23:4938-4944.). BARF1 protein can complex in vitro with CSF1 (Colony Stimulating Factor-1) in resulting in the inhibition of macrophage activation (Strockbine L D, Cohen J I, Farrah T, Lyman S D, Wagener F, DuBose R F, Armitage R J and Spriggs M K L D. 1998, J Virol. 72: 4015-4021.), and can also inhibit the secretion of INF-alpha in EBV-infected B cells (Cohen J. and Lekstrom K. 1999 J. Virol. 73: 7627-7632). On the other hand, BARF1 was recognized by NK cells in ADCC test (Tanner J, Wei M, Ahamad A, Alfieri C, Tailor P, Ooka T, Menezes J. 1997 J. Infect Disease. 175: 38-46.). BARF1 is therefore involved not only in oncogenic mechanism, but also in immunomodulation. BARF1 has an anti-apoptotic activity when expressed in gastric epithelial cells (Wang Q., Tsao S W., Ooka T., Nicholls J M., Cheung H W, Fu S., Wong Y C, Wang X. 2006 Cancer Letter 238 :90-103). BARF1 activates cell cycle by its autocrine mechanism in activating several cellular proteins involved in anti-apoptosis. Its mitogenic activity plays a key role in the development of EBV-related tumor (Sall A, Caserta S, Jolicoeur P, Franqueville L, de Turenne-Tessier M, Ooka T. 2004, Oncogene 23: 4938-44).

As BARF1 and LMP1-exosome are secreted in culture medium (Houali K, X. Wang, Y. Shimizu, D. Djennaoui, J. Nicholls, S. Fiorini, A. Bougermouh and T. Ooka. 2007, Clin. Cancer Res. 13: 4993-5000), these oncoproteins were also secreted in serum as well as in saliva from NPC patients. These secreted proteins in serum can play crucial role for cell activation, immunomodulation and/or immunosuppression. The secretion of BARF1 protein in serum and saliva of NPC patients was demonstrated (Houali K, X. Wang, Y. Shimizu, D. Djennaoui, J. Nicholls, S. Fiorini, A. Boμguermouh and T. Ooka. 2007, Clin. Cancer Res. 13: 4993-5000) and BARF1 protein showed powerful mitogenic activity in vitro. This mitogenic activity would be related to the development of tumor.

The role of BARF1 as an oncogene required for the immortalization of B cells has been described. However, other oncogenes have been described and are required for immortalization.

EP-A-1 229 043 describes different peptides derived from LMP1, LMP2 and BARF1 and antibody reagents reactive therewith. However, immunotherapy assays using these antibodies are not described. Further, the peptides described in EP-A-1 229 043 comprise between 34 and 42 amino acids.

Decaussin et al. (Cancer Res., 60:5584-8, 2000) describe peptides derived from BARF1 and antibodies raised against these peptides. These antibodies were used to detect expression of BARF1 in Nasopharyngeal carcinoma.

However, in the state of the art, immunotherapy has never been applied successfully with antibodies binding specifically to BARF1.

The present invention surprisingly shows that antibodies binding specific regions of BARF1 are capable of neutralising the oncoprotein in vivo allowing prevention and suppression of tumors in a mouse model.

Polyclonal antibodies binding to three peptides derived from BARF1 were produced. Successive injection of anti-BARF1 antibodies before injection of NPC-derived epithelial tumor cells led to prevention of tumor apparition. When anti-BARF1 antibodies were successively injected after the tumor size became about 0.8 cm in diameter, the tumor regressed and completely disappeared. This represents the first report on immunotherapy with anti-LMP1 antibodies suppressing and protecting from EBV positive tumors.

Addition of anti-BARF1 antibodies into culture medium was also able to inhibit EBV-positive B cell growth, suggesting that immunotherapy based on anti-BARF1 is also efficient to inhibit and protect from the development of EBV-associated lymphomas.

Treatment and prevention based on immunotherapy by anti-BARF1 is efficient not only for NPC type-carcinoma, but also GC type-carcinoma Inhibitory effect was observed in vivo and in vitro.

Immunotherapy based on anti-BARF1 antibody treatment seems all the more promising as anti-BARF1 antibodies are almost undetectable in the serum of NPC patients.

### SEQUENCE LISTING

SEQ ID No. 1: Peptide derived from BARF1 corresponding to positions N172 to D180 of BARF1 protein from Human Herpesvirus 4 type 1 (Genebank: Gene ID 3783772, mRNA and protein YP_{—}401719.1)
SEQ ID No. 2: Peptide derived from BARF1 corresponding to positions G203 to E209 of BARF1 protein from Human Herpesvirus 4 type 1 (Genebank: Gene ID 3783772, mRNA and protein YP_{—}401719.1)
SEQ ID No. 3: Peptide derived from BARF1 corresponding to positions W48 to E55 of BARF1 protein from Human Herpesvirus 4 type 1 (Genebank: Gene ID 3783772, mRNA and protein YP_{—}401719.1)

### DESCRIPTION OF THE INVENTION

A first object of the present invention is a composition for use as a medicament comprising an antibody or an antibody fragment binding specifically to a peptide selected from SEQ ID Nos. 1-3.

In a preferred embodiment, the composition for use as a medicament comprises an antibody or an antibody fragment binding specifically to the peptide of SEQ ID No. 1.

In another preferred embodiment, the composition for use as a medicament comprises an antibody or antibody fragment that is a monoclonal antibody, a chimeric antibody or a humanised antibody.

A second object of the present invention is a composition for use as a medicament or as a vaccine comprising a peptide selected from SEQ ID Nos. 1-3.

Preferably, the composition for use as a medicament or as a vaccine comprises the peptide of SEQ ID No. 1.

Another object of the present invention is a composition for use as a medicament or as a vaccine comprising a polynucleotide encoding a peptide selected from SEQ ID Nos. 1-3.

The invention also relates to compositions for use as a medicament or as a vaccine comprising a transformed host cell expressing a peptide selected from SEQ ID Nos. 1-3.

Preferably, the compositions are for prevention or treatment of Epstein-Barr Virus positive tumors.

More preferred, the compositions are for prevention or treatment of nasopharyngeal carcinoma, gastric carcinoma, Burkitt's lymphoma, Hodgkin's lymphoma, lymphoma induced in AIDS patients, esophage and intrahepatic cholangiocarcinoma, nasal NK/T-cell lymphoma and oral hairy leucoplasia (OHL).

Even more preferred, the compositions are for prevention or treatment of nasopharyngeal carcinoma or of gastric carcinoma.

The present invention relates to compositions for use as a medicament comprising an antibody or antibody fragment binding specifically to selected peptides from EBV protein BARF1. The present invention further relates to compositions for use as a medicament or as a vaccine comprising selected peptides derived from EBV protein BARF1. Another object of the present invention is a composition for use as a medicament or as a vaccine comprising a polynucleotide encoding the selected peptides of BARF1. A further object of the present invention is a composition for use as a medicament or as a vaccine comprising a transformed host cell expressing a selected peptide derived from BARF1.

Peptides derived from BARF1 and antibodies reagents reactive thereto had already been described but had never been used successfully in immunotherapy. It has now been surprisingly found that antibodies binding specifically to the peptides of SEQ ID Nos. 1-3 are able to prevent and reduce tumor development in an in vivo mouse model.

The present invention provides pharmaceutical compositions comprising:
a) an effective amount of an antibody or antibody fragment as described herein, an effective amount of a pepetide as described herein, an effective amount of a polynucleotide as described herein or an effective amount of a transformed host cell as described herein, and
b) a pharmaceutically acceptable carrier, which may be inert or physiologically active.

The present invention further provides vaccine compositions comprising:
a) an effective amount of a polypeptide as described herein, an effective amount of a polynucleotide as described herein, or an effective amount of a transformed host cell as described herein and
b) an adjuvant.

As used herein, “pharmaceutically-acceptable carriers” includes any and all solvents, dispersion media, coatings, antibacterial and antifungal agents, and the like that are physiologically compatible. Examples of suitable carriers, diluents and/or excipients include one or more of water, saline, phosphate buffered saline, dextrose, glycerol, ethanol, and the like, as well as combination thereof. In many cases, it will be preferable to include isotonic agents, such as sugars, polyalcohols, or sodium chloride in the composition. In particular, relevant examples of suitable carrier include: (1) Dulbecco's phosphate buffered saline, pH ˜7.4, containing or not containing about 1 mg/ml to 25 mg/ml human serum albumin, (2) 0.9% saline (0.9% w/v sodium chloride (NaCl)), and (3) 5% (w/v) dextrose; and may also contain an antioxidant such as tryptamine and a stabilizing agent such as Tween 20.

The pharmaceutical compositions encompassed by the present invention may also contain a further therapeutic agent for the treatment of cancers associated with EBV.

The compositions of the invention may be in a variety of forms. These include for example liquid, semi-solid, and solid dosage forms, but the preferred form depends on the intended mode of administration and therapeutic application. Typical preferred compositions are in the form of injectable or infusible solutions. The preferred mode of administration is parenteral (e.g. intravenous, intramuscular, intraperinoneal, subcutaneous). In a preferred embodiment, the compositions of the invention are administered intravenously as a bolus or by continuous infusion over a period of time. In another preferred embodiment, they are injected by intramuscular, subcutaneous, intra-articular, intrasynovial, intratumoral, peritumoral, intralesional, or perilesional routes, to exert local as well as systemic therapeutic effects.

Sterile compositions for parenteral administration can be prepared by incorporating the antibody, the antibody fragment, the polypeptide, or the polynucleotide as described in the present invention in the required amount in the appropriate solvent, followed by sterilization by microfiltration. As solvent or vehicle, there may be used water, saline, phosphate buffered saline, dextrose, glycerol, ethanol, and the like, as well as combination thereof. In many cases, it will be preferable to include isotonic agents, such as sugars, polyalcohols, or sodium chloride in the composition. These compositions may also contain adjuvants, in particular wetting, isotonizing, emulsifying, dispersing and stabilizing agents. Sterile compositions for parenteral administration may also be prepared in the form of sterile solid compositions which may be dissolved at the time of use in sterile water or any other injectable sterile medium.

The antibody, antibody fragment, polypeptide, polynucleotide or transformed host cell as described herein may also be orally administered. As solid compositions for oral administration, tablets, pills, powders (gelatine capsules, sachets) or granules may be used. In these compositions, the active ingredient according to the invention is mixed with one or more inert diluents, such as starch, cellulose, sucrose, lactose or silica, under an argon stream. These compositions may also comprise substances other than diluents, for example one or more lubricants such as magnesium stearate or talc, a coloring, a coating (sugar-coated tablet) or a glaze.

As liquid compositions for oral administration, there may be used pharmaceutically acceptable solutions, suspensions, emulsions, syrups and elixirs containing inert diluents such as water, ethanol, glycerol, vegetable oils or paraffin oil. These compositions may comprise substances other than diluents, for example wetting, sweetening, thickening, flavoring or stabilizing products.

The doses depend on the desired effect, the duration of the treatment and the route of administration used.

The invention is also related to the use of an antibody, antibody fragment, polypeptide, polynucleotide or transformed host cell as described herein for the manufacture of a medicament or for the manufacture of a vaccine for the prevention or treatment of EBV positive tumors or EBV associated tumors such as nasopharyngeal carcinoma, gastric carcinoma, Burkitt's lymphoma, Hodgkin's lymphoma, lymphoma induced in AIDS patients, esophage and intrahepatic cholangiocarcinoma, nasal NK/T-cell lymphoma and oral hairy leucoplasia (OHL).

In a preferred embodiment, antibodies, antibody fragments, polypeptides, polynucleotides or transformed host cells as described herein, are used for prevention or treatment of EBV positive tumors. In a more preferred embodiment, one of the pharmaceutical or vaccine compositions disclosed above, and which contains an antibody, antibody fragment, polypeptide, polynucleotide or transformed host cell as described herein, is used for prevention or treatment of EBV positive tumors. More preferably, they are used for prevention or treatment of nasopharyngeal carcinoma, gastric carcinoma, Burkitt's lymphoma, Hodgkin's lymphoma, lymphoma induced in AIDS patients, esophage and intrahepatic cholangiocarcinoma, nasal NK/T-cell lymphoma and oral hairy leucoplasia (OHL). In a preferred embodiment, they are used for prevention or treatment of nasopharyngeal carcinoma or of gastric carcinoma.

The present invention also provides methods for preventing or treating EBV positive tumors including administering an effective amount of an antibody, antibody fragment, polypeptide, polynucleotide, transformed host cell as described herein to a human or to a patient in need thereof. In a preferred embodiment, the invention relates to methods for prevention or treatment of nasopharyngeal carcinoma, gastric carcinoma, Burkitt's lymphoma, Hodgkin's lymphoma, lymphoma induced in AIDS patients, esophage and intrahepatic cholangiocarcinoma, nasal NK/T-cell lymphoma and oral hairy leucoplasia (OHL). Even more preferred, the invention relates to methods for prevention or treatment of nasopharyngeal carcinomaor of gastric carcinoma.

In a first embodiment, the compositions of the present invention comprise an antibody or an antibody fragment binding specifically to selected peptides from BARF1.

As used herein the term “binding” refers to an antibody or antibody fragment that reacts with an epitope of one of the peptides of SEQ ID Nos. 1-3 or that was raised against one of the peptides of SEQ ID Nos. 1-3.

Preferably, the antibody binds specifically to an epitope of one of the peptides of SEQ ID Nos. 1-3 and does not crossreact with other antigens. Thus, the antibody reacts with one specific antigen.

In the present invention, polyclonal antibodies binding specifically to peptides of SEQ ID Nos. 1-3 were produced in rabbits. Polyclonal or monoclonal antibodies binding specifically to the peptides of SEQ ID Nos. 1-3 may be produced by standard techniques. Preferred antibodies are antibodies binding to the peptide of SEQ ID No. 1.

The term “antibody” is used herein in the broadest sense and specifically covers monoclonal antibodies of any isotype such as IgG, IgM, IgA, IgD and IgE, polyclonal antibodies, chimeric antibodies, humanized antibodies and antibody fragments. An antibody reactive with a specific antigen can be generated by recombinant methods such as selection of libraries of recombinant antibodies in phage or similar vectors, or by immunizing an animal with the antigen or an antigen-encoding nucleic acid.

A typical IgG antibody is comprised of two identical heavy chains and two identical light chains that are joined by disulfide bonds. Each heavy and light chain contains a constant region and a variable region. Each variable region contains three segments called “complementarity-determining regions” (“CDRs”) or “hypervariable regions”, which are primarily responsible for binding an epitope of an antigen. They are usually referred to as CDR1, CDR2, and CDR3, numbered sequentially from the N-terminus. The more highly conserved portions of the variable regions are called the “framework regions”.

As used herein, “VH” or “VH” refers to the variable region of an immunoglobulin heavy chain of an antibody, including the heavy chain of an Fv, scFv, dsFv, Fab, Fab′ or F(ab′)2 fragment. Reference to “VL” or “VL” refers to the variable region of the immunoglobulin light chain of an antibody, including the light chain of an Fv, scFv, dsFv, Fab, Fab′ or F(ab′)2 fragment.

A “polyclonal antibody” is an antibody which was produced among or in the presence of one or more other, non-identical antibodies. In general, polyclonal antibodies are produced from a B-lymphocyte in the presence of several other B-lymphocytes producing non-identical antibodies. Usually, polyclonal antibodies are obtained directly from an immunized animal.

A “monoclonal antibody”, as used herein, is an antibody obtained from a population of substantially homogeneous antibodies, i.e. the antibodies forming this population are essentially identical except for possible naturally occurring mutations which might be present in minor amounts. These antibodies are directed against a single epitope and are therefore highly specific.

An “epitope” is the site on the antigen to which an antibody binds.

As used herein, a “chimeric antibody” is an antibody in which the constant region, or a portion thereof, is altered, replaced, or exchanged, so that the variable region is linked to a constant region of a different species, or belonging to another antibody class or subclass. “Chimeric antibody” also refers to an antibody in which the variable region, or a portion thereof, is altered, replaced, or exchanged, so that the constant region is linked to a variable region of a different species, or belonging to another antibody class or subclass. Methods for producing chimeric antibodies are known in the art.

The term “humanized antibody”, as used herein, refers to a chimeric antibody which contain minimal sequence derived from non-human immunoglobulin. The goal of humanization is a reduction in the immunogenicity of a xenogenic antibody, such as a murine antibody, for introduction into a human, while maintaining the full antigen binding affinity and specificity of the antibody. Humanized antibodies, or antibodies adapted for non-rejection by other mammals, may be produced using several technologies such as resurfacing and CDR grafting. Humanized chimeric antibodies preferably have constant regions and variable regions other than the complementarity determining regions (CDRs) derived substantially or exclusively from the corresponding human antibody regions and CDRs derived substantially or exclusively from a mammal other than a human.

The antibodies of the present invention include both the full length antibodies discussed above, as well as epitope-binding fragments thereof. As used herein, “antibody fragments” include any portion of an antibody that retains the ability to bind to the epitope recognized by the full length antibody, generally termed “epitope-binding fragments.” Examples of antibody fragments include, but are not limited to, Fab, Fab′ and F(ab′)2, Fd, single-chain Fvs (scFv), single-chain antibodies, disulfide-linked Fvs (dsFv) and fragments comprising either a VL or VH region. Epitope-binding fragments, including single-chain antibodies, may comprise the variable region(s) alone or in combination with the entirety or a portion of the following: hinge region, CH1, CH2, and CH3 domains.

In a second embodiment, the compositions of the present invention comprise a peptide selected from the peptides of SEQ ID Nos. 1-3.

In a third embodiment, the compositions of the present invention comprise a polynucleotide encoding a pepetide selected from the peptides of SEQ ID Nos. 1-3.

The term “polynucleotide” according to the present invention refers to a single strand nucleotide chain or its complementary strand which can be of the DNA or RNA type, or a double strand nucleotide chain which can be of the cDNA (complementary) or genomic DNA type. Preferably, the polynucleotides of the invention are of the DNA type, namely double strand DNA. The term “polynucleotide” also refers to modified polynucleotides.

The polynucleotides of this invention are isolated or purified from their natural environment. Preferably, the polynucleotides of this invention can be prepared using conventional molecular biology techniques such as those described by Sambrook et al. (Molecular Cloning: A Laboratory Manual, 1989) or by chemical synthesis.

The polynucleotide may be a vector such as for example a viral vector.

Another object of the invention is a composition comprising a transformed host cell expressing a peptide selected from the peptides of SEQ ID Nos. 1-3.

The man skilled in the art is well aware of the standard methods for incorporation of a polynucleotide into a host cell, for example transfection, lipofection, electroporation, microinjection, viral infection, thermal shock, transformation after chemical permeabilisation of the membrane or cell fusion.

### FIGURES

FIG. 1: Effect of anti-BARF1 antibodies in EBV positive or EBV negative cell lines:

5 μg of PepIII antibodies for 10⁵ cells were added in the culture of human EBV-positive AKATA and EBV-negative Louckes B cell lines as well as human epithelial c666-1 cell line, then cultured during 120 hours. Survival % was determined by Coommassi blue staining.

FIG. 2: Effect of anti-BARF1 Pep III antibodies on EBV-AGS Cell growth

EBV-negative AGS (1) and EBV-positive AGS (2) were cultured in the presence of 5 μg of Pep III antibody. Control cells did not receive antibody. Cell viability was measured by coommassi blue staining during 5 days.

FIG. 3: Immunotherapy assay on nasopharyngeal carcinoma-derived tumor:

50 μg of anti-BARF1 PepIII antibody was intrapenetorially injected before (j), simultaneously (k) or after injection of c666-1 cells (l). 10⁷ cells (c666-1) were injected subcutaneously. The value presented in the figure correspond to the average tumor size diameter in mm. Protocol 1: (j) with S12 for c666-1. Protocol 2: (k) with S12 for c666-1. Protocol 3: (l) with S12 for c666-1. Tumor development after injection of c666-1 cells without any antibody (i).

FIG. 4: Immunotherapy assay on gastric carcinoma-derived tumor

Anti-BARF1 PepIII was injected before (n), simultaneously (o) or after injection of EBV-AGS (p) cells. 50 μg of antibody were injected intrapenetorially. 10⁷ cells (EBV-AGS) were injected subcutaneously. The value presented in the figure correspond to average of tumor size in a diameter of mm. Protocol 1: (n) with PepIII for EBV-AGS. Protocol 2: (o) Effect of Pep III on EBV-AGS cell growth with PepIII for EBV-AGS. Protocol 3: (p) with PepIII for EBV-AGS. Tumor development after injection of AGS-EBV cells without any antibody (m).

FIG. 5: Effect of anti-EBV DNAase in tumor development

Rabbit polyclonal Anti-EBV DNAase (50 μg) was treated every 5 days during 20 days, then 10⁶ c666-1 cells were injected. Tumor development was monitored. no inhibitory effect of antidody on tumor development.

FIG. 6: Effect of anti-rabbit Ig on tumor development

Anti-rabbit Ig or anti-mouse Ig (50 μg) were treated every 5 days during 20 days, then 10⁶ c666-1 cells were injected. Tumor development was monitored. no inhibitory effect of antidody on tumor development.

FIG. 7: Detection of BARF1/PepIII complex in mouse serum and tumor cells by immunoblot BARF1/PepIII complex was isolated and analysed on 12% SDS-polyacrylamide gel. Antigen antibody complexes were detected with an enhanced chemiluminescence system (ECL; Amersham). The presence of BARF1 were analyzed in serum from mice developing c666-1 or EBV-AGS tumor (1). Control positives were: purified BARF1 p29. BARF1/PepIII complex isolated from serum: (2) S-c666-1. BARF1/PepIII complex isolated from tumor (3): MT-c666-1. Pep III was revealed by secondary rabbit anti-Ig). Commercial rabbit Ig was used as positive control: Ig (1, 2, 3).

### EXAMPLES

To demonstrate that anti-BARF1 antibody can be used for prevention and suppression of EBV-associated carcinomas (NPC and GC), we used an animal model: nude mice.

Polyclonal antibodies binding to the peptides of SEQ ID No. 1-3 were produced in rabbits.

Polyclonal antibodies, hereafter called PEPIII, binding to the peptide of SEQ ID No. 1 were produced in rabbits as described previously (Decaussin et al., Cancer Res., 60:5584-8, 2000).

For in vitro analysis of the effect of anti-BARF1 antibody, polyclonal anti-BARF1 was examined in EBV-positive NPC-derived c666-1 epithelial cell line and EBV-positive or EBV-negative human B cell lines. NPC-derived or GC-derived tumor could be induced when NPC-derived c666-1 epithelial cells (Cheung S T, Huang D P, Hui A B, Lo K W, Ko C W, Tsang Y S, Wong N, Whitney B M, Lee J C. 1999, Int J Cancer 83:121-6) or GC-derived EBV-positive AGS epithelial cells (Kassis J, Maeda A, Teramoto N, Takada, K, Wu C, Wells A. 2002, Int. J. Cancer 99: 644-51) were injected into nude mice. We analysed the effect of anti-BARF1 in these mice.

### In Vitro Experiment

### Effect of Anti-BARF1 (PEPIII Antibodies) on Cell Culture

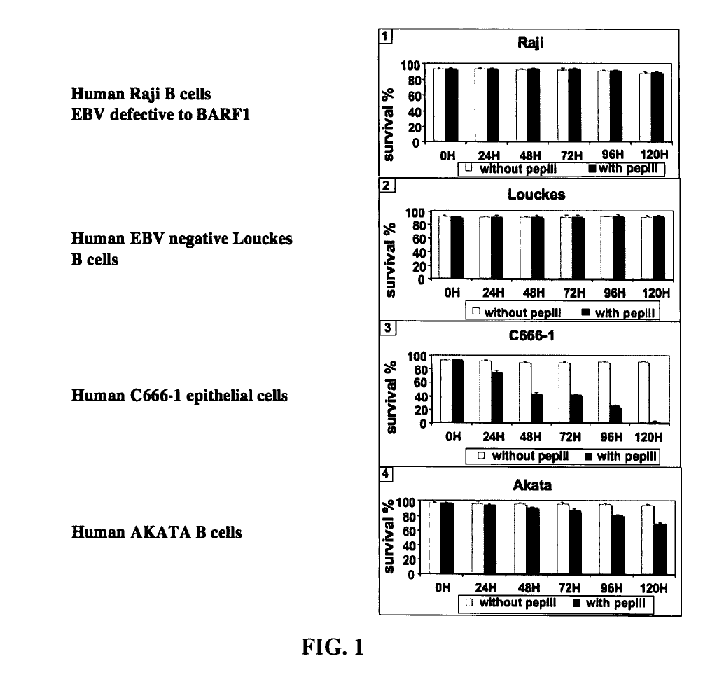

Effect of anti-BARF1 antibody was analysed on EBV-positive c666-1 epitheial cell line, EBV-positive human AKATA and Raji B cell lines, EBV-negative human Louckes B cell line, human gastric AGS cell line and EBV-positive human gastric AGS cell line in culture in vitro (FIG. 1).

The c666-1 cells secreted BARF1 protein into the culture medium (Houali K, X. Wang, Y. Shimizu, D. Djennaoui, J. Nicholls, S. Fiorini, A. Bouguermouh and T. Ooka. 2007, Clin. Cancer Res. 13: 4993-5000).

When this secreted oncoprotein was neutralised by anti-BARF1 antibody in vitro (5 μg of antibody for 10⁵ cells added in culture medium), the c666-1 cells went to die as presented by survial curve in the FIG. 1. After addition of antibody, the survival cells diminished to 75% at 24 h, 40% at 48 h, 20% at 96 h and almost all c666-1 cells went to die after 120 minutes (5 days) (FIG. 1-3). This suggests that mitogenic activity of BARF1 is directly related to main cell activation process.

Similar phenomenon was observed in EBV-positive human AKATA B cells, but with lesser effect. At 120 min, about 30% of cells went to die as presented in FIG. 1-4. This phenomenon is probably related a low level of BARF1 expression in these cells (Fiorini and Ooka, 2008 Virology J. 5 :70).

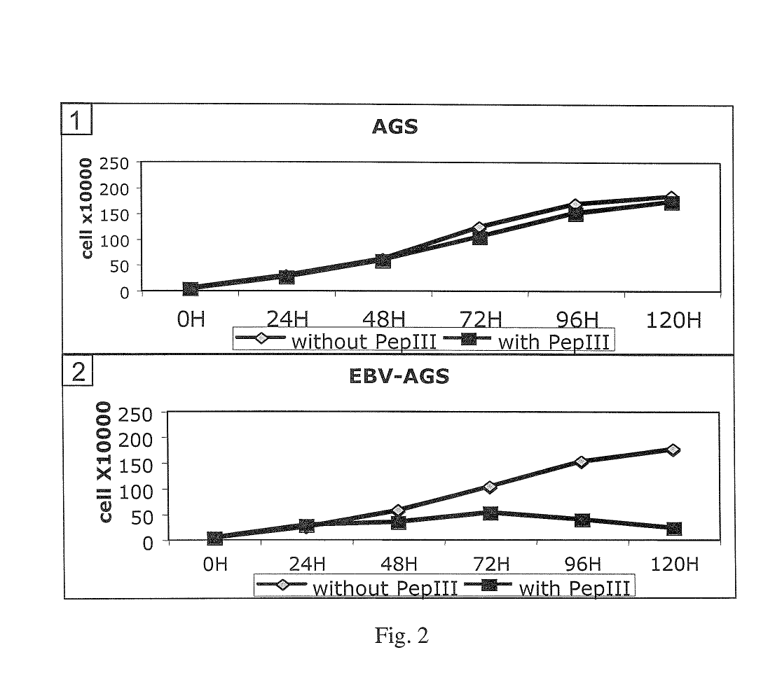

As expected, no inhibitory effect was observed on EBV-negative Louckes B cell (FIG. 1-2) and EBV-positive Raji B cell in where Raji EBV genome is negative to BARF1 sequence (FIG. 1-1).

The inhibitory effect of anti-BARF1 was examined on gastric carcinoma's AGS or EBV-AGS cells (FIG. 2). Addition of anti-BARF1 in culture medium did not show any inhibition on AGS cell growth (FIG. 2-1), while anti-BARF1 gave significant inhibition of EBV-AGS cell growth at 120 h. At this time, almost all EBV-AGS cells went to die (FIG. 2-2). This inhibition came from BARF1 protein secreted in culture of EBV-AGS. In conclusion, anti-BARF1 antibody which recognize the epitope 172-180th a.a. of BARF1 protein could inhibit cell growth of EBV-positive c666-1 and AGS epithelial cell lines and EBV-positive B cell line expressing BARF1 protein.

### In Vivo Experiment

We investigated the activity of anti-BARF1 antibody in nude mice injected subcutaneously with 10⁷ cultured EBV-associated tumor cell lines: c666-1 cell derived from NPC or EBV-positive AGS derived from GC (EBV-AGS). Nude mice used here come from Harlan (France) produced in Italy: Strain: Hsd :Athymic Nude-Fox1^{nu}. We also tested HsdCpb :NMRI-Fox1^{nu}. Their age is 4 weeks. Their sex is male. Their weight at 4 weeks is about 19-21 g.

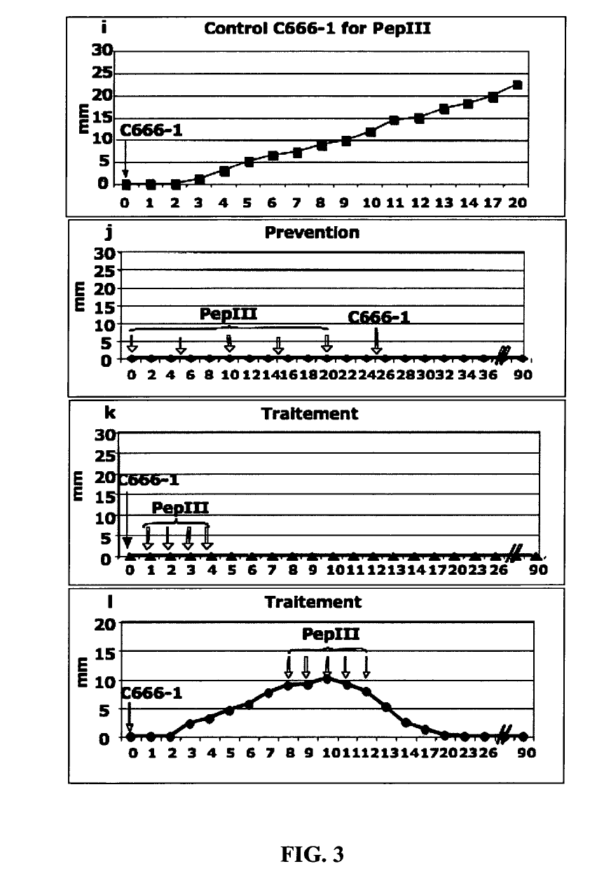

With c666-1 cells, tumor is detectable in untreated mice by the second or third day, reaches a diameter of ca. 2 mm by day 4, and 8 mm at day 8, then 16 mm at day 14 and 20 mm at 20 days (FIG. 3-i).

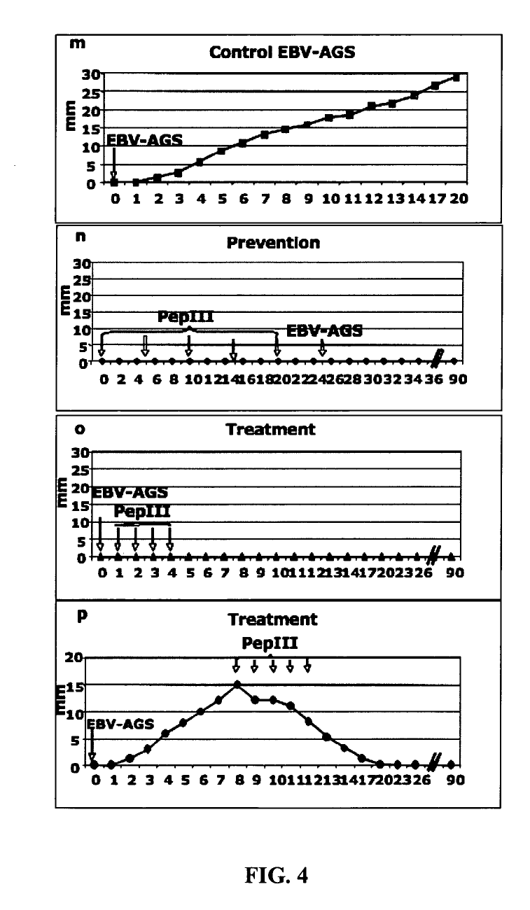

With EBV-positive AGS cells, tumor is detectable in untreated mice by the second or third day, reaches a diameter of ca. 3 mm by day 4, and 15 mm at day 8, then 25 mm at day 14 and 30 mm at 20 days (FIG. 4-m).

Induced tumors (tumor size in mm in diameter) are slightly smaller with c666-1 cells than with EBV-AGS cells (FIG. 3-i and FIG. 4-m).

To analyse the effect of anti-BARF1, 25 μg of anti-BARF1 per mice was injected by intraperitoneal way in three protocols:

Protocol #1, BARF1 (Pep-III) was administered as 5 intraperitoneal injections of 25 μg at 5 day intervals finishing 3 days before tumor challenge in the preventive protocol (FIG. 3-j for c666-1 and FIG. 4-n for EBV-AGS)
Protocol #2, 5 successive daily injections starting either simultaneously with tumor challenge (FIG. 3-k for c666-1 and FIG. 4-o for EBV-AGS).
Protocol #3, 5 injections (one injection everyday) when the tumor size became about 0.8 cm in diameter (FIG. 3-4 for c6666-1 and FIG. 4-4 for EBV-AGS).
Protocol #1 and #2 are for prevention and protocol #3 is tumor treatment.

Preventive (protocol #1—FIG. 3-j and FIG. 4-n) or simultaneous (protocol #2—FIG. 3-k and FIG. 4-o) treatment with anti-BARF1 for both cell lines completely abrogated tumor appearance in any of the treated mice for at least 3 months.

Injection of anti-BARF1 antibody was also highly effective if given when the tumors had already reached a considerable size. Nodules of ca. 8 mm (c666-1) and ca. 15 mm (EBV-AGS) rapidly stabilized, then regressed progressively after treatment by 5 daily injections of anti-BARF1 antibody (FIG. 3-1 for c666-1 and FIG. 4-p for EBV-AGS). The tumor masses disappeared completely at 11 days after onset of treatment, and the mice remained tumor-free for at least 3 months.

To confirm the specificity of anti-BARF1 on the inhibition of tumor growth, we injected either EBV-encoded DNAase antibody or mouse polyclonal anti-Ig antibody in Protocol #1 (Preventive). Either anti-EBV-DNAase or anti-mouse Ig was administered as 5 intraperitoneal injections of 25 μg at 5 day intervals finishing 3 days before tumor challenge in the preventive protocol.

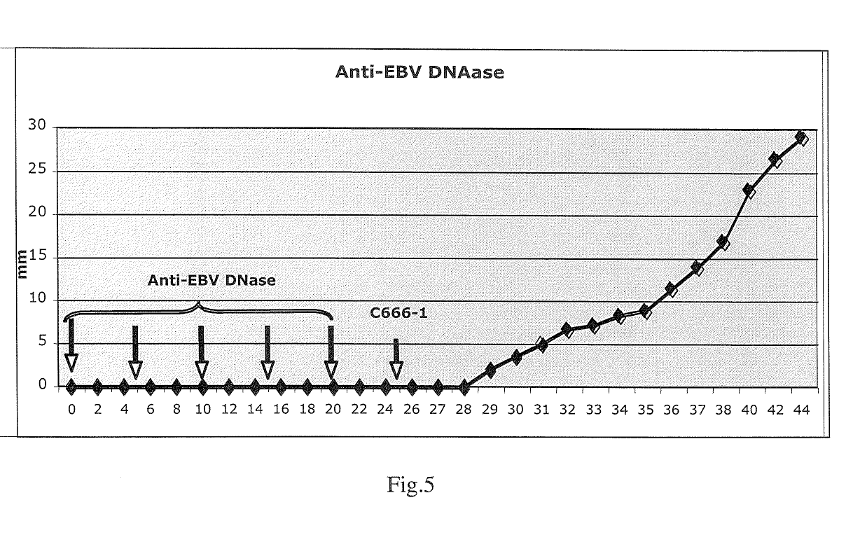

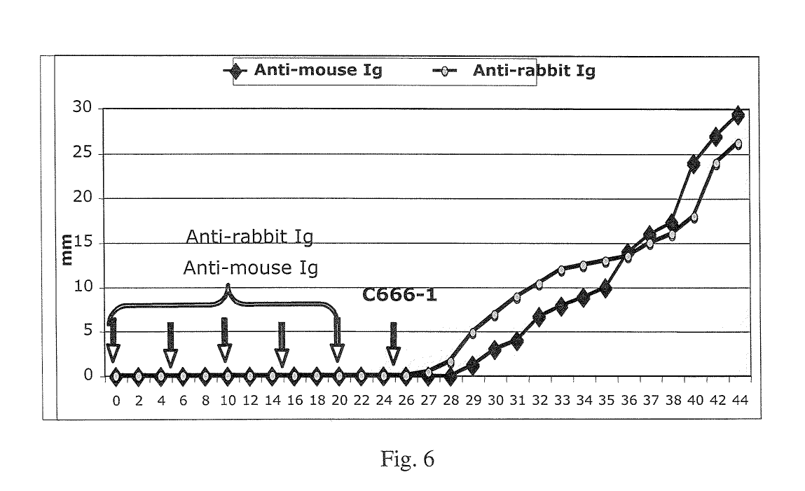

When untreated or treated animals with anti-DNAase in protocol #1 (preventive) with c666-1 (FIG. 5) or with anti-rabbit Ig (FIG. 6) in the place of anti-BARF1 used as control experiment showed rapid tumor growth (FIG. 5 and FIG. 6). This suggests that the specific inhibition of tumor development is probably due to neutralisation of BARF1 protein by anti-BARF1.

Anti-rabbit Ig used was purchased from Sigma (France) Cat. N^{o} 18772.

Rabbit polyclonal anti-DNAase used here was produced in our laboratory from EBV-DNAase obtained by Baculovirus system (Sbih-Lammali F, Berger F, Busson P and Ooka T, 1996, Virology, 222: 64-74) (Zeng Y, Middeldorp J, Madjar J J and Ooka T, 1997, Virology 239:285-295).

We then examined if the complex of anti-BARF1 and BARF1 protein is present in serum as well as in tumor cells.

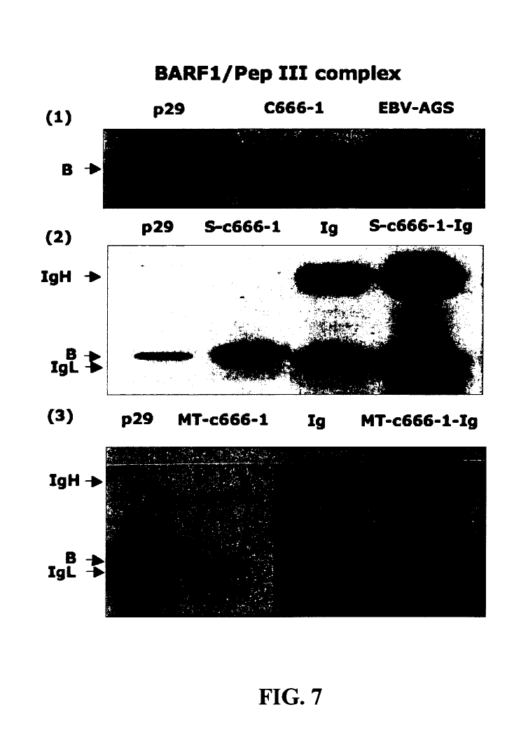

BARF1 was present in the serum of mice bearing c666-1 (FIG. 7-1, c666-1) and EBV-AGS (FIG. 7-1. EBV-AGS). Positive control used in this experiment (called p29) come from BARF1 purified from 293 cell infected by BARF1 recombinant adenovirus (Sall A, Caserta S, Jolicoeur P, Franqueville L, De Turenne-Tessier M and Ooka T. (2004) Oncogene. 23:4938-4944).

We investigated these serum components in mice treated with antibody after the development of tumor (protocol #3). BARF1 was recovered by concanavalin A affinity chromatography from serum on the third day of treatment (Houali K, X. Wang, Y. Shimizu, D. Djennaoui, J. Nicholls, S. Fiorini, A. Bougermouh and T. Ooka. Clin. Cancer Res. 13: 4993-5000; De Turenne and Ooka, 2007 J. Gen. Virol. 88: 2656-2661).

Analysis of complex in serum of c666-1-treated mice by Western blot shows the presence of BARF1 (FIG. 7-2: S-c666-1) associated with rabbit immunoglobulin (FIG. 7-2, S-c666-1-Ig). Commercial rabbit Ig was added as a control positive (FIG. 7-2: Ig).

Similar complexes were also present in tumor biopsies (FIG. 7-3, MT-c666-1) in association with rabbit immunoglobulin (FIG. 7-3, MT-c666-1-Ig). Commercial mouse Ig was added as a control positive (FIG. 7-3: Ig).

Surprisingly, we found the BARF1/PepIII antibody complexes inside of cells isolated from tumor biopsies from the appropriately treated mice. Cells extracted from tumor were layered out on slide and fixed with aceton. The complex was revealed by anti-rabbit Ig for PepIII. BARF1/PepIII complexes were seen as intracytoplasmic and intranuclear patches. Apparently, these usually mitogenic components were rendered ineffective through combination with its specific antibodies.

We have previously shown that BARF1 purified from serum of NPC patients was mitogenic in EBV-negative Louckes cells (Houali K, X. Wang, Y. Shimizu, D. Djennaoui, J. Nicholls, S. Fiorini, A. Bougermouh and T. Ooka. Clin. Cancer Res. 13: 4993-5000).

In general, almost all EBV-positive GC biopsies express BARF1 (zur Hausen A, Brink A A, Craanen M E., Middeldorp J M, Meijer C J, van Den Brule A J. 2000, Cancer Res. 60: 2745-2748). Transcription of BARF1 was compared in c666-1 and EBV-AGS cells ex vivo and in culture by semi-quantitative and quantitative RT-PCR.

We found that BARF1 expression is somehow weak in cultured c666-1 cells, but its expression becomes much more important in tumor biopsies. For EBV-AGS, BARF1 transcription was very weak in cultured EBV-AGS cells, but tumor biopsies express much more BARF1. As the actin expression used as standard control was similar between them, these suggest that BARF1 expression was activated ex vivo. Activation of EBV genes expression ex vivo was already observed by our group when analysed in tumor biopsie after injection of EBV-AKATA cells in nude mice (Sheng W, Decaussin, G., Ligout A., Takada, K and Ooka, T. 2003. J. Virol. 77: 3859-3865).

We confirmed these results by quantitative RT-PCR. Transcription level was increased two folds in EBV-AGS tumor (EBV-AGS/EBV-AGS-T) and almost three folds in c666-1 tumor (c666-1/c666-1-T) in comparison with the value obtained from cultured cells (EBV-AGS and c666-1).

Spectacular activation of BARF1 transcription in tumor suggests that its important role in cell cycle activation to develop tumor.

1. A method for preventing or treating Epstein-Barr Virus positive tumors comprising administering an effective amount of an antibody or an antibody fragment binding specifically to a peptide selected from SEQ ID Nos. 1-3 to a patient in need thereof.

2. A method according to claim 1 wherein the antibody or an antibody fragment binds specifically to the peptide of SEQ ID No. 1.

3. A method of claim 1, wherein the antibody or antibody fragment is a monoclonal antibody, a chimeric antibody or a humanised antibody.

4. A method for preventing or treating Epstein-Barr Virus positive tumors comprising administering an effective amount of a peptide selected from SEQ ID Nos. 1-3 to a patient in need thereof.

5. A method according to claim 4 comprising the peptide of SEQ ID No. 1.

6. A method for preventing or treating Epstein-Barr Virus positive tumors comprising administering an effective amount of a polynucleotide encoding a peptide selected from SEQ ID Nos. 1-3 to a patient in need thereof.

7. A method for preventing or treating Epstein-Barr Virus positive tumors comprising administering an effective amount of a transformed host cell expressing a peptide selected from SEQ ID Nos. 1-3 to a patient in need thereof.

8. (canceled)

9. (canceled)

10. (canceled)

11. (canceled)

12. Method for preventing or treating Epstein-Barr Virus positive tumors according to claim 1, wherein the Epstein-Barr Virus positive tumor is selected from the group consisting of nasopharyngeal carcinoma, gastric carcinoma, Burkitt's lymphoma, Hodgkin's lymphoma, lymphoma induced in AIDS patients, esophage and intrahepatic cholangiocarcinoma, nasal NK/T-cell lymphoma and oral hairy leucoplasia (OHL).

13. Method for preventing or treating Epstein-Barr Virus positive tumors according to claim 4, wherein the Epstein-Barr Virus positive tumor is selected from the group consisting of nasopharyngeal carcinoma, gastric carcinoma, Burkitt's lymphoma, Hodgkin's lymphoma, lymphoma induced in AIDS patients, esophage and intrahepatic cholangiocarcinoma, nasal NK/T-cell lymphoma and oral hairy leucoplasia (OHL).

14. Method for preventing or treating Epstein-Barr Virus positive tumors according to claim 6, wherein the Epstein-Barr Virus positive tumor is selected from the group consisting of nasopharyngeal carcinoma, gastric carcinoma, Burkitt's lymphoma, Hodgkin's lymphoma, lymphoma induced in AIDS patients, esophage and intrahepatic cholangiocarcinoma, nasal NK/T-cell lymphoma and oral hairy leucoplasia (OHL).

15. Method for preventing or treating Epstein-Barr Virus positive tumors according to claim 7, wherein the Epstein-Barr Virus positive tumor is selected from the group consisting of nasopharyngeal carcinoma, gastric carcinoma, Burkitt's lymphoma, Hodgkin's lymphoma, lymphoma induced in AIDS patients, esophage and intrahepatic cholangiocarcinoma, nasal NK/T-cell lymphoma and oral hairy leucoplasia (OHL).

16. A composition comprising an antibody or an antibody fragment binding specifically to a peptide selected from SEQ ID Nos. 1-3
